(19) United States
(12) Patent Application Publication
(10) Pub. No.: US 2006/0292202 A1
(43) Pub. Date: Dec. 28, 2006

(54) Drug delivery device

(75) Inventor: Stephen P. Bartels, Pittsford, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/167,484

(22) Filed: Jun. 27, 2005

(51) Int. Cl. A61K 31/4745; A61F 2/00

(52) U.S. Cl. 424/427; 514/291

(57) ABSTRACT

Drug delivery devices include a beta-carboline active agent for treatment of ophthalmic disorders.

### FIELD OF THE INVENTION

This invention relates to compositions and devices for delivering a pharmaceutically active agent including a beta-carboline to the eye, as well as methods employing such compositions for treating an ophthalmic disorder.

### BACKGROUND OF THE INVENTION

US Patent Application Publication 2004-0102438-A1, the disclosure of which is incorporated herein by reference, discloses the use of beta-carbolines for the treatment of neurogenerative diseases of the eye. The preferred compositions have the form of eye drops, ointments, gels, or tablets. The beta-carbolines have GABA-receptor-modulating activity (GABA denoting gamma-amino-butyric acid).

### SUMMARY OF THE INVENTION

This invention provides compositions and drug delivery devices for delivering a beta-carboline active agent to the eye, and/or for treating an ophthalmic disorder. Additionally, the invention relates to methods employing such compositions. The beta-carboline active agent is delivered locally to eye tissue, and preferably in a sustained release manner, so that relatively small doses of the active are exposed to eye tissue over an extended period of time.

According to a first embodiment, the drug delivery device comprises a polymeric material and a beta-carboline. The may have the form of a prefabricated solid matrix of the polymeric material loaded with the beta-carboline. The polymeric material of this matrix may include a hydrogel copolymer, such as a silicone hydrogel copolymer, or a non-hydrogel silicone polymer.

According to another embodiment, the device may comprise a drug core that includes the beta-carboline, and a holder comprising the polymeric material, wherein the drug core is held in the holder. The holder may comprise an impermeable polymer that is impermeable to said active agent, where the holder includes at least one opening for passage of the pharmaceutically agent therethrough. The drug core may comprise a mixture of the beta-carboline and a permeable polymeric material that is permeable to said active agent. As an example, the permeable polymeric material comprises poly(vinyl alcohol) and the holder comprises a silicone-containing polymer. The holder may comprises a cylinder that surrounds the drug core, in which case the device may include a suture tab attached to said cylinder for suturing the device to eye tissue.

According to other embodiments, the device comprises a matrix of a polymeric material and a beta-carboline.

The devices of this invention may be implanted in eye tissue, sutured to eye tissue, and/or injected in eye tissue.

### BRIEF DESCRIPTION OF THE DRAWING FIGURES

FIG. 1 is a perspective view of a first embodiment of a drug delivery device of this invention.

FIG. 2 is a cross-sectional view of the device of FIG. 1.

FIG. 3 is a cross-sectional view of the device of FIGS. 1 and 2 during assembly.

### DETAILED DESCRIPTION OF VARIOUS PREFERRED EMBODIMENTS

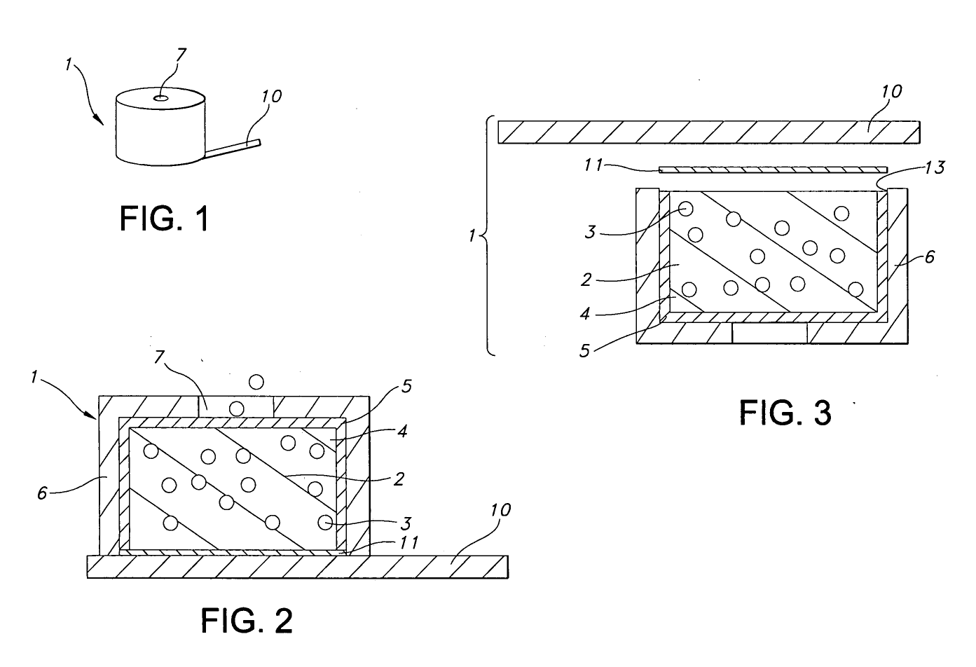

According to a first embodiment, the beta-carboline may be contained in the holder of a drug delivery device. An example of such a device is shown in FIGS. 1 and 2. Device 1 is a sustained release drug delivery device for implanting in the eye. Device 1 includes inner drug core 2 including a pharmaceutically active agent 3 inclusive of the beta-carboline. As shown in the illustrated embodiment, active agent 3 may be mixed with a polymeric material 4. Material 4 is a polymeric material that is compatible with body fluids and the eye. Additionally, this material should be permeable to passage of the active agent 3 therethrough, particularly when the device is exposed to body fluids. For the illustrated embodiment, this polymeric material is poly(vinyl alcohol) (PVA). Also, in this embodiment, inner drug core 2 may be coated with a coating 5 of additional polymeric material which may be the same or different from material 4 mixed with the active agent. For the illustrated embodiment, the coating 5 employed is also PVA.

Device 1 includes a holder 6 for the inner drug core 2. Holder 6 is made of a material that is impermeable to passage of the active agent 3 therethrough. Since holder 6 is made of the impermeable material, at least one passageway 7 is formed in holder 6 to permit active agent 3 to pass therethrough and contact eye tissue. In other words, upon exposure to body fluids, active agent passes through any permeable material 4 and permeable coating 5, and exits the device through passageway 7. For the illustrated embodiment, the holder is made of silicone, especially polydimethylsiloxane (PDMS) material.

A device of the type shown in FIGS. 1 and 2 may be assembled by the following procedures, referring also to FIG. 3. A cylindrical cup of silicone is provided, having a size generally corresponding to the drug core tablet and a shape as generally shown in FIG. 2, and including openings 7. A drop of liquid PVA is placed into the holder through the end 13 of the holder. Then, the inner drug core tablet is placed into the silicone holder through the same end 13 and pressed into the cylindrical holder. As a result, the pressing of the tablet causes the liquid PVA to fill the space between the tablet inner core and the silicone holder, thus forming permeable layer 5 shown in FIGS. 1 and 2. A layer of adhesive 11 may be applied to the end 13 of the holder to fully enclose the inner drug core tablet at this end. Suture tab 10 is inserted at this end of the device. The liquid PVA and adhesive may be cured by heating the assembly.

It will be appreciated the dimensions of the device can vary with the size of the device, the size of the inner drug core, and the holder that surrounds the core or reservoir. The physical size of the device should be selected so that it does not interfere with physiological functions at the implantation site of the mammalian organism. The targeted disease state, type of mammalian organism, location of administration, and agents or agent administered are among the factors which would effect the desired size of the sustained release drug delivery device. However, because the device is intended for placement in the eye, the device is relatively small in size. Generally, it is preferred that the device, excluding the suture tab, has a maximum height, width and length each no greater than 15 mm, more preferably no greater than 10 mm, and most preferably no greater than 5 mm.

Many other configurations of sustained release drug delivery devices may be used for the delivery of the beta-carboline. Examples are found in the following patent literature, the disclosures of which are incorporated herein by reference: US 2002/0086051A1 (Viscasillas); US 2002/0106395A1 (Brubaker); US 2002/0110591A1 (Brubaker et al.); US 2002/0110592A1 (Brubaker et al.); US 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Mosack et al.); and U.S. patent application Ser. No. 11/006,914 (filed Dec. 8, 2004, Kunzler et al.).

As mentioned, the illustrated embodiment includes a tab 10 in order to attach the device to a desired location in the eye, for example, by suturing. Alternately, the device may omit a suture tab extension and be implanted surgically without suturing. Additionally, the sustained release device may be injected into eye tissue, for example, by insertion into the vitreous through a 0.5-mm opening in the sclera provided by a TSV-25 cannula.

The amount of beta-carboline active agent included in the device may vary. For example, in the case where the device is intended to release the active agent over a longer period, a higher amount of beta-carboline would be used than if the device was intended for a shorter period of release. Generally, for the illustrated embodiment, the beta-carboline active agent will be included in the drug core 2 in an amount of 0.1 to 10% (w/w), more preferably, 1 to 5% (w/w), based on total weight of the drug core matrix.

According to other embodiments, the drug delivery compositions comprise a solid matrix of a polymeric material and the beta-carboline pharmaceutically active agent.

This matrix material may be formed into a desired shape, such as a film, sphere, cylinder or lens-shaped article. The resultant device may be implanted surgically in the eye, for example, the drug delivery device may be implanted below the sclera. Alternately, the device may be implanted by injecting the device into the eye. For example, a sphere- or cylinder-shaped matrix may be inserted into the vitreous through a 0.5-mm opening in the sclera provided by a TSV-25 cannula. This prefrabricated solid device will be sized and shaped for delivery to eye tissue, and it is preferred that the device has a maximum height, width and length each no greater than 15 mm, more preferably no greater than 10 mm, and most preferably no greater than 5 mm. Generally, for this embodiment, the active agent is included in the polymeric matrix in an amount of 0.1 to 10% (w/w), more preferably, 1 to 5% (w/w), based on total weight of the matrix.

As a first example, the polymeric material may be a silicone hydrogel loaded with the pharmaceutically active agent.

A hydrogel is a hydrated crosslinked or insolubilized polymeric system that contains water in an equilibrium state. Hydrogel devices are generally formed by polymerizing a mixture of device-forming monomers including at least one hydrophilic monomer. Hydrophilic device-forming monomers include: unsaturated carboxylic acids such as methacrylic acid and acrylic acid; (meth)acrylic substituted alcohols or glycols such as 2-hydroxyethyl methacrylate, 2-hydroxyethyl acrylate, and glyceryl methacrylate; vinyl lactams such as N-vinyl-2-pyrrolidone; and acrylamides such as methacrylamide and N,N-dimethylacrylamide. Other hydrophilic monomers are well-known in the art.

The monomer mixture generally includes a crosslinking monomer, a crosslinking monomer being defined as a monomer having multiple polymerizable functionalities. One of the hydrophilic monomers may function as a crosslinking monomer or a separate crosslinking monomer may be employed. Representative crosslinking monomers include: divinylbenzene, allyl methacrylate, ethylene glycol dimethacrylate, tetraethyleneglycol dimethacrylate, polyethyleneglycol dimethacrylate, and vinyl carbonate derivatives of the glycol dimethacrylates.

In the case of silicone hydrogels, the device-forming monomer mixture includes, in addition to a hydrophilic monomer, at least one silicone-containing monomer. When the silicone-containing monomer includes multiple polymerizable groups, it may function as the crosslinking monomer. This invention is particularly suited for extraction of silicone hydrogel biomedical devices. Generally, unreacted silicone-containing monomers, and oligomers formed from these monomers, are hydrophobic and more difficult to extract from the polymeric device.

One suitable class of silicone containing monomers include known bulky, monofunctional polysiloxanylalkyl monomers represented by Formula (I):

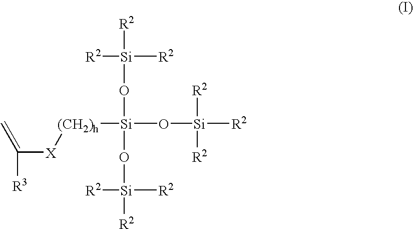

X denotes —COO—, —CONR⁴—, —OCOO—, or —OCONR⁴— where each where R⁴ is H or lower alkyl; R³ denotes hydrogen or methyl; h is 1 to 10; and each R² independently denotes a lower alkyl or halogenated alkyl radical, a phenyl radical or a radical of the formula
—Si(R⁵)₃
wherein each R⁵ is independently a lower alkyl radical or a phenyl radical. Such bulky monomers specifically include 3-methacryloxypropyltris(trimethylsiloxy)silane, pentamethyldisiloxanylmethyl methacrylate, methyldi(trimethylsiloxy)methacryloxymethylsilane, 3-[tris(trimethylsiloxy)silyl]propylvinyl carbamate, and 3-[tris(trimethylsiloxy)silyl]propylvinyl carbonate.

Another suitable class is multifunctional ethylenically “end-capped” siloxane-containing monomers, especially difunctional monomers represented Formula (II): 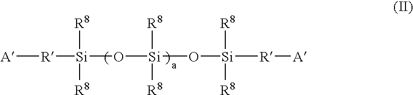
wherein:

each A′ is independently an activated unsaturated group;

each R′ is independently are an alkylene group having 1 to 10 carbon atoms wherein the carbon atoms may include ester, ether, urethane or ureido linkages therebetween;

each R⁸ is independently selected from monovalent hydrocarbon radicals or halogen substituted monovalent hydrocarbon radicals having 1 to 18 carbon atoms which may include ether linkages therebetween, and

a is an integer equal to or greater than 1. Preferably, each R⁸ is independently selected from alkyl groups, phenyl groups and fluoro-substituted alkyl or alkyloxy groups. It is further noted that at least one R⁸ may be a fluoro-substituted alkyl group such as that represented by the formula:
-D′-(CF₂)_{S}-M′
wherein:

D′ is an alkylene group having 1 to 10 carbon atoms wherein said carbon atoms may include ether linkages therebetween;

M′ is hydrogen, fluorine, or alkyl group but preferably hydrogen; and

s is an integer from 1 to 20, preferably 1 to 6.

With respect to A′, the term “activated” is used to describe unsaturated groups which include at least one substituent which facilitates free radical polymerization, preferably an ethylenically unsaturated radical. Although a wide variety of such groups may be used, preferably, A′ is an ester or amide of (meth)acrylic acid represented by the general formula: 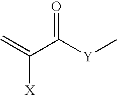
wherein X is preferably hydrogen or methyl, and Y is —O— or —NH—. Examples of other suitable activated unsaturated groups include vinyl carbonates, vinyl carbamates, fumarates, fumaramides, maleates, acrylonitryl, vinyl ether and styryl. Specific examples of monomers of Formula (II) include the following: 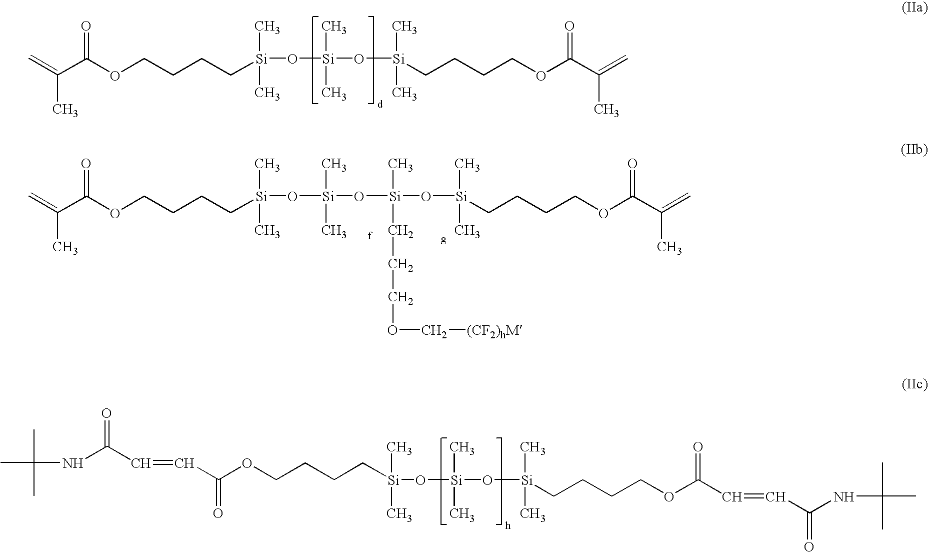
wherein:

d, f, g and k range from 0 to 250, preferably from 2 to 100; h is an integer from 1 to 20, preferably 1 to 6; and

M′ is hydrogen or fluorine.

A further suitable class of silicone-containing monomers includes monomers of the Formulae (IIIa) and (IIIb):
E′(*D*A*D*G)ₐ*D*A*D*E′; or (IIIa)
E′(*D*G*D*A)ₐ*D*G*D*E′; (IIIb)
wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureido linkage;

a is at least 1;

A denotes a divalent polymeric radical of the formula: 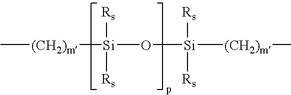
wherein:

each R^{S} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each E′ independently denotes a polymerizable unsaturated organic radical represented by the formula: 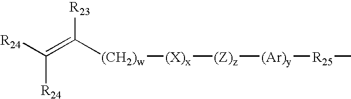
wherein:

R₂₃ is hydrogen or methyl;

R₂₄ is hydrogen, an alkyl radical having 1 to 6 carbon atoms, or a —CO—Y—R₂₆ radical wherein Y is —O—, —S— or —NH—;

R₂₅ is a divalent alkylene radical having 1 to 10 carbon atoms; R₂₆ is a alkyl radical having 1 to 12 carbon atoms; X denotes —CO— or —OCO—; Z denotes —O— or —NH—; Ar denotes an aromatic radical having 6 to 30 carbon atoms; w is 0 to 6; x is 0 or 1; y is 0 or 1; and z is 0 or 1.

A specific urethane monomer is represented by the following: 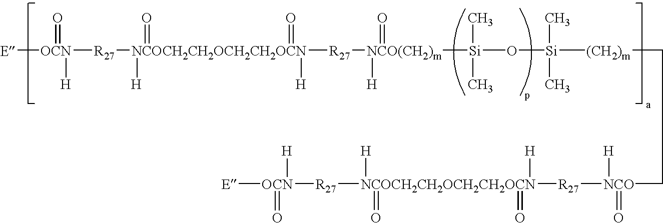
wherein m is at least 1 and is preferably 3 or 4, a is at least 1 and preferably is 1, p is a number which provides a moiety weight of 400 to 10,000 and is preferably at least 30, R₂₇ is a diradical of a diisocyanate after removal of the isocyanate group, such as the diradical of isophorone diisocyanate, and each E″ is a group represented by:

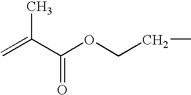

Other silicone-containing monomers include the silicone-containing monomers described in U.S. Pat. Nos. 5,034,461, 5,070,215, 5,260,000, 5,610,252 and 5,496,871, the disclosures of which are incorporated herein by reference. Other silicone-containing monomers are well-known in the art.

These matrices of a silicone hydrogel and active agent may be prepared by mixing the active agent and the device-forming monomeric mixture, including any diluent. Then, this initial mixture is added to a mold providing the final shape and configuration of the solid matrix device. While contained in the mold, the mixture is polymerized by exposure to light energy, such as a UV light source, or a source of visible light in the blue spectrum. Alternately, the mixture may be cured thermally. Finally, the resultant solid matrix device is recovered from the mold, and subjected to any desired post-molding operation, such as extraction to remove impurities, packaging, and sterilization.

As a second example, the polymeric material may be a silicone-containing, non-hydrogel polymer loaded with the pharmaceutically active agent. This class of materials include at least one silicone-containing monomer as the device-forming monomer. A crosslinking monomer may also be included in the initial monomeric mixture, although when the silicone-containing monomer includes multiple polymerizable radicals, it may function as the crosslinking monomer. Additionally, this initial monomeric mixture may include a non-silicone hydrophobic co-monomer, such as an alkyl(meth)acrylate or fluoroalkyl(meth)acrylate.

The pharmaceutically active agent is added to the device-forming monomeric mixture, including any diluent, and this initial mixture is added to a mold providing the final shape and configuration of the solid matrix device. While contained in the mold, the mixture is polymerized by exposure to light energy and/or thermal energy. The resultant solid matrix device is removed from the mold and extracted with a solvent, packaged and sterilized.

Particularly suitable beta-carbolines include the following, as well as pharmaceutically suitable salts thereof: abecarnil; 3,4-dihydro-beta-carboline; gedocamil; 1-methyl-1-vinyl-2,3,4-trihydro-beta-carboline-3-carboxylic acid; 6-methoxy-1,2,3,4-tetrahydro-beta-carboline; N—BOC-L-1,2,3,4-tetrahydro-beta-carboline-3-carboxylic acid; tryptoline; pinoline; methoxyharmalan; tetrahydro-beta-carboline (THBC); 1-methyl-THBC; 6-methoxy-THBC; 6-hydroxy-THBC; 6-methoxyharmalan; norharman; and 3,4-dihydro-beta-carboline.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate and the like.

The drug delivery devices containing the beta-carbolines are used to treat ophthalmic disorders, including diseases of the retina. These include treatment of vascular diseases of the retina, such as retinopathia angiospastica, arteriosclerotic retinopathy, eclamptic retinopathy, diseases caused by occlusions of the aorta carotis, periphlebitis retinae, diabetic retinopathy, non-proliferative diabetic retinopathy, proliferative diabetic retinopathy, diabetic maculopathy, carcinoma-associated retinopathy and/or retinopathy due to radiation trauma. The devices can be used to treat diseases caused by venous and/or arterial vascular occlusions, such as diseases caused by branch vein occlusions, central vein occlusion, arterial occlusion, amaurosis fugax, occlusion of venule of retina, chronic ocular ischemia, sickle cell retinopathy, ocular ischemic syndrome and/or retinitis exsudativa. The devices can be used for the treatment of macular degenerations, such as moist and dry macular degeneration, acquired macular degenerations, age-related macular degeneration, retinopathia centralis serosa, myopic macular changes, cystiform macular edema, vasiform stripes, toxic macular diseases, maculaforamen, exudative maculopathies due to other causes, chlorioretinopathy centralis serosa, cystiform macular edema, submacular bleeding, hereditary macular and retinal degenerations, juvenile macular degenerations, vitelline macular degenerations, albinism, storage diseases, amaurotic idiocy, sphingolipidoses, Tay-Sachs disease, Niemann-Pick disease, gangliosidosis, Gaucher's disease, Spielmeyer-Vogt-Stock disease and/or in Sandhoffs disease. The devices can be used in the treatment of traumatic retinal changes such as contusion of the eye, perforating eye injuries, siderosis/hemidosis, chalcosis, burns, retinopathia traumatica and/or injury to the retina from light. In addition, the devices can be used for treatment of retinoschisis, of diseases of the choroid, such as hyalin deposits and/or choroideremia, and of diseases of the optic nerve, such as trauma to the nervus opticus caused by intoxications such as tobacco-alcohol trauma, trauma caused by methyl alcohol, trauma caused by ethambutol, trauma caused by quinine, arsenic, lead and/or bromine. The devices can also be used for anterior ischemic optic neuropathy, such as apoplexia papillae and/or Horton's syndrome, and treatment of an optic atrophy, such as traumatic optic atrophy, optic atrophy caused by tumour pressure, hereditary optic atrophy, liver optic atrophy, secondary optic atrophy, optic atrophy after papillitis/retrobulbar neuritis, optic atrophy of uncertain origin, glaucomatous optic atrophy and/or changes to the optic nerve head. The devices can be used for treatment of glaucoma, such as primary glaucoma, Donders' glaucoma, primary Donders' glaucoma, normotension glaucoma, angle-closure glaucoma, acute angle-closure glaucoma, intermittent angle-closure glaucoma, subacute angle-closure glaucoma, chronic angle-closure glaucoma, plateau iris and/or nanopthhalmos. The devices can be used for congenital glaucoma and premature glaucoma, such as cornea-angle of chamber-iris dysgeneses, Lowe's syndrome, Sturge-Weber syndrome, neurofibromatosis, Rubinstein-Taybi syndrome, Pierre Rubin syndrome, Ota's nevus, trisomy, Marfan syndrome, Turner's syndrome, aniridia, homocystinuria, intraocular tumours, orbital lymphangioma, retinopathia praematurorum, persistent hyperplastic primary vitreous body, ectopia lensis, intraocular inflammation, cortisone therapy, myopia with pigmentary glaucoma, rubella embryopathy, cataract extraction and/or for treatment of blunt or acute trauma. The devices can be used for treatment of glaucoma simplex, such as glaucoma with aphakia and pseudoaphakia, glaucoma with diabetes mellitus, glaucoma and dystrophia endotheliasis, hypersecretion glaucoma, glaucoma in pregnancy, higher myopia and/or juvenile glaucoma, and for the treatment of secondary glaucoma, such as traumatic and postoperative glaucoma, secondary Donders' glaucoma, secondary angle-closure glaucoma, steroid-induced glaucoma, glaucoma after inflammation, phacolytic glaucoma, Posner-Schlossman syndrome, heterochromic cyclitis, ghost cell glaucoma, hemolytic glaucoma, neurofibromatosis, siderosis, glaucoma caused by regeneration of vessels, glaucoma caused by cortisone administration, pigmentary glaucoma, pseudoexfoliation glaucoma, glaucoma with anterior uveitis, glaucoma with Fuchs heterochromia, Grant's syndrome, glaucoma after contusions, chamber angle abnormalities of non-traumatic origin, erythroclastic glaucoma, silcione glaucoma, lens-related glaucoma, phacotopical glaucoma, phacomorphic glaucoma, glaucoma caused by free lens material, pseudoexfoliation glaucoma, phacogenic uveitis, glaucoma with anterior uveitis, malignant glaucoma and/or for glaucoma caused by increased episcleral venous pressure. Also, the devices can be used to treat ocular hypertension, for example for the primary and secondary form. As used herein, “treatment” and like terms include administering the beta-carboline-containing device to a subject, including mammals such as humans, in need thereof, including: to delay progression of the ophthalmic disorder; to prevent damage to eye tissue; to delay progression of damage to eye tissue; and so forth.

The examples and illustrated embodiments demonstrate some of the sustained release embodiments of the present invention. However, it is to be understood that these examples are for illustrative purposes only and do not purport to be wholly definitive as to the conditions and scope. While the invention has been described in connection with various preferred embodiments, numerous variations will be apparent to a person of ordinary skill in the art given the present description, without departing from the spirit of the invention and the scope of the appended claims.

What I claim is:

1. A drug delivery device for placement in the eye, comprising a polymeric material and a beta-carboline.

2. The drug delivery device of claim 1, including at least one beta-carboline selected from the group consisting of: abecamil; 3,4-dihydro-beta-carboline; gedocarnil; 1-methyl-1-vinyl-2,3,4-trihydro-beta-carboline-3-carboxylic acid; 6-methoxy-1,2,3,4-tetrahydro-beta-carboline; N—BOC-L-1,2,3,4-tetrahydro-beta-carboline-3-carboxylic acid; tryptoline; pinoline; methoxyharmalan; tetrahydro-beta-carboline (THBC); 1-methyl-THBC; 6-methoxy-THBC; 6-hydroxy-THBC; 6-methoxyharmalan; norharman; 3,4-dihydro-beta-carboline; and a pharmaceutically suitable salt thereof.

3. The drug delivery device of claim 1, wherein the device is a prefabricated solid matrix of the polymeric material loaded with the beta-carboline.

4. The drug delivery device of claim 3, wherein the prefabricated solid has a maximum height, width and length each no greater than 15 mm.

5. The drug delivery device of claim 3, wherein the beta-carboline is released from the matrix in a sustained manner.

6. The drug delivery device of claim 3, wherein the polymeric material comprises a hydrogel copolymer.

7. The drug delivery device of claim 3, wherein the polymeric material comprises a silicone hydrogel copolymer.

8. The drug delivery device of claim 3, wherein the polymeric material comprises a non-hydrogel silicone polymer.

9. The drug delivery device of claim 1, comprising a drug core that includes the beta-carboline, and a holder comprising the polymeric material, wherein the drug core is held in the holder.

10. The drug delivery device of claim 1, wherein the holder comprises an impermeable polymer that is impermeable to said active agent.

11. The drug delivery device of claim 10, wherein the holder includes at least one opening for passage of the pharmaceutically agent.

12. The drug delivery device of claim 9, wherein the drug core comprises a mixture of the beta-carboline and a permeable polymeric material that is permeable to said active agent.

13. The drug delivery device of claim 12, wherein the permeable polymeric material comprises poly(vinyl alcohol) and the holder comprises a silicone-containing polymer.

14. The drug delivery device of claim 9, wherein the holder comprises a cylinder that surrounds the drug core.

15. The drug delivery device of claim 14, wherein the device includes a suture tab attached to said cylinder for suturing the device to eye tissue.

16. The drug delivery device of claim 9, wherein the drug core is coated with a material permeable to said active agent.

17. The drug delivery device of claim 1, wherein the device comprises abecarnil or a pharmaceutically acceptable salt thereof.

18. A method of treating ophthalmic disorders, comprising administering to a patient a drug delivery device of claim 1.

19. The method of claim 18, wherein the beta-carboline is released from the polymeric material in a sustained manner.

20. The method of claim 18, wherein the device comprises abecarnil or a pharmaceutically acceptable salt thereof.

21. The method of claim 18, wherein the device is implanted in eye tissue.

22. The method of claim 18, wherein the device is injected in eye tissue.

23. The method of claim 18, wherein the device is implanted at a back portion of the eye.

24. A method comprising delivering to eye tissue a composition comprising a matrix of a polymeric material and a beta-carboline.

25. The method of claim 24, wherein the beta-carboline is released from the matrix in a sustained manner.
